(19) United States
(12) Patent Application Publication
(10) Pub. No.: US 2012/0021504 A1
(43) Pub. Date: Jan. 26, 2012

(54) AERATED COMPOSTER AND WASTE COLLECTION BIN

(76) Inventor: Michael Bradlee, Providence, RI (US)

(21) Appl. No.: 13/187,184

(22) Filed: Jul. 20, 2011

Related U.S. Application Data
(60) Provisional application No. 61/365,878, filed on Jul. 20, 2010.

(51) Int. Cl. C12M 1/00

(52) U.S. Cl. 435/290.4; 435/290.1

(57) ABSTRACT

An aerated compost and waste collection bin is disclosed. The bin includes a body portion having a bottom, at least one sidewall from the bottom, and an open top forming a container. A perforated tube extends upwards from the bottom of the body portion. At least one lateral vent tube is connected to the perforated tube and the sidewall of the body portion. The lateral vent tube allows air to circulate from outside the body portion of the bin through to the perforated tube.

### CROSS-REFERENCE TO RELATED APPLICATION

This application claims priority to earlier filed U.S. Provisional Application Ser. No. 61/365,878, filed Jul. 20, 2010, the entire contents of which are incorporated herein by reference.

### BACKGROUND OF THE INVENTION

1. Field of the Invention

The present patent document relates generally to composting and waste collection and more specification to an aerated composter and organic waste collection bin.

2. Background of the Related Art

Composting has many benefits to the environment. Compost is a key ingredient in organic farming. At its most essential, the process of composting requires simply piling up waste outdoors and waiting a year or more.

Modern, methodical composting is a multi-step, closely monitored process with measured inputs of water, air and carbon- and nitrogen-rich materials. The decomposition process is aided by shredding the plant matter, adding water and ensuring proper aeration by regularly turning the mixture. Worms and fungi further break up the material. Aerobic bacteria manage the chemical process by converting the inputs into heat, carbon dioxide and ammonium. The ammonium is further converted by bacteria into plant-nourishing nitrites and nitrates through the process of nitrification.

Compost can be rich in nutrients. It is used in gardens, landscaping, horticulture, and agriculture. The compost itself is beneficial for the land in many ways, including as a soil conditioner, a fertilizer, addition of vital humus or humic acids, and as a natural pesticide for soil.

In ecosystems, compost is useful for erosion control, land and stream reclamation, wetland construction, and as landfill cover (see compost uses). Compost can also be used to generate biogas through anaerobic digestion.

In closely packed commercial or residential settings, however, composting can become problematical. Specifically, the matter that is decaying into compost can emanate offensive odors. Furthermore, liquid is released from the decomposition of material composting which can make a mess. The liquid, called “compost tea”, is a liquid solution or suspension made by steeping compost in water. It is used as both a fertilizer and in attempts to prevent plant diseases. It is therefore desirable to collect this compost tea for use

Therefore, there is a perceived need in the art to create a compost bin that minimizes odors of composting material, thus making the system desirable in suburban and urban settings. There is also a perceived need for a compost bin that enables the easy collection of compost tea.

### SUMMARY OF THE INVENTION

The compost bin of the present invention includes a container that has a perforated, dual ventilation tube design that penetrates into the center of the bin, and thus the composting materials, and allows for more thorough air circulation, and aeration of composting materials. The container's ventilation is placed strategically to minimize or eliminate any leakage from the unit while still allowing efficient and thorough air exchange. Whereas most compost bins contain perforations on the lower part of the shell, and thus lack control of leakage; this tubular design ventilated materials from top to bottom, and eliminates the need for shell ventilation holes at the bottom half of the bin; this design allows for complete containment of liquids and controlled liquids release via the spigot at the base of the bin.

The perforated vertical ventilation tube attached to dual lateral vent tubes allows for continuous air flow and more frequent air exchange and thus oxygenation of composting materials; air exchange and oxygenation of composting materials accelerates the composting process and minimizes odor events associated with anaerobic conditions and stagnant air. The perforated vertical tubing is sized to allow for efficient air exchange while mitigating material's clogging the vent system. The lateral tubes are vented on the underside of the tubes; this also allows for efficient air exchange while preventing materials from entering the perforations and clogging the ventilation. The dual vent system allows for continuous flow of air through the vent system; as opposed to a single vent that would compete with air flow entering and exiting the vent system. With an option for a powered fan attached to the upper vertical tube, air exchange and removal of excess condensation are further enhanced and composting is further accelerated as a result. Power sources for the fan may be battery or a solar photovaltaic cell.

The vertical ventilation also allows for liquids to filter through perforations and via gravity action collect in the bottom of the compost bin. A drain with a spigot at the base of the compost bin is fastened via an inverted half-pipe to the floor of the bin and to the flange of the vertical tube. By attaching the drain system to the flange of the perforated ventilation tube, this allows for efficient control and removal of liquids from the compost bin. These liquids are valuable and may be collected and converted into a compost tea; which may be utilized as a liquids fertilizer and foliar spray.

This method to remove liquids and excess condensation from the compost bin (via enhanced air circulation through the vent tubes and the drain system) also allows for overall weight reduction of materials, which may result in cost reductions associated with tipping the bins during servicing at a commercial site.

These bins are designed to be utilized in at a commercial site as well as a residential dwelling. These compost bins are tippable and may be integrated into a waste and recycling management system. These compost bins contain wheels that allow for ease of movement and placement. The vertical upright design results in a small footprint of the unit.

These bins are designed for extending collection and storage for Source Separated Organics while minimizing odors associated with waste food materials. These compost bins are intended to be utilized at an institution such as a school or hospital, a commercial site, such as a restaurant or university dining hall or a residential dwelling. These compost bins are also designed to allow for accelerated and efficient composting and thus the composting process may be initiated at any time during the collection process. By allowing the option to initiate the composting process at the collection site, material's collection time is lengthened and odor events are minimized and or eliminated. Longer collection windows translates into less frequent tipping and servicing and lower costs associated with waste and recycling management.

### BRIEF DESCRIPTION OF THE DRAWINGS

These and other features, aspects, and advantages of the present invention will become better understood with reference to the following description, appended claims, and accompanying drawings where:

FIG. 1 is a partially exploded, cross-section view from the front, right and top sides of an embodiment of the aerated composter and waste collection bin of the present invention;

FIG. 2 is a rear elevation view of an embodiment of the aerated composter and waste collection bin of the present invention;

FIG. 3 is a partially exploded, cross-section view from the front, right and top sides of a first alternative embodiment of the aerated composter and waste collection bin of the present invention that includes a top and bottom sorting shelves;

FIG. 4 is a partial cross-section view from the front, right and top sides of a second alternative embodiment of the aerated composter and waste collection bin of the present invention showing an alternate orientation of the drain;

FIG. 5 is a partial cross-section view from the front, right and top sides of a third alternative embodiment of the aerated composter and waste collection bin of the present invention showing an alternate orientation of the drain that also includes a top and bottom sorting shelves like the second alternative embodiment;

FIG. 6 is a the front, right and top sides of a fourth alternative embodiment of the aerated composter and waste collection bin of the present invention showing an access door on the lower right side of the alternative embodiment;

FIG. 7a is a the front, right and top sides of a fifth alternative embodiment of the aerated composter and waste collection bin of the present invention showing an alternative access door on the lower right side of the alternative embodiment with a removable basket; and

FIG. 7b is a close up view of the fifth alternative embodiment shown in FIG. 5b, which exhibits the removable basket being extracted from the access door.

### DESCRIPTION OF THE PREFERRED EMBODIMENT

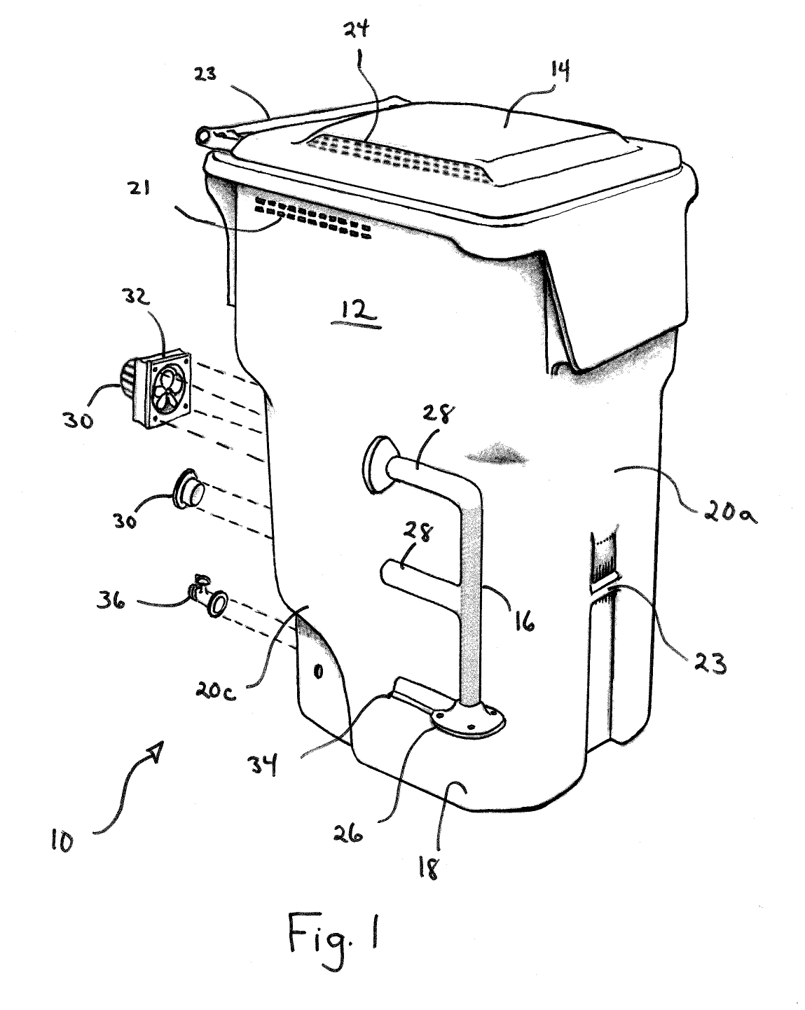

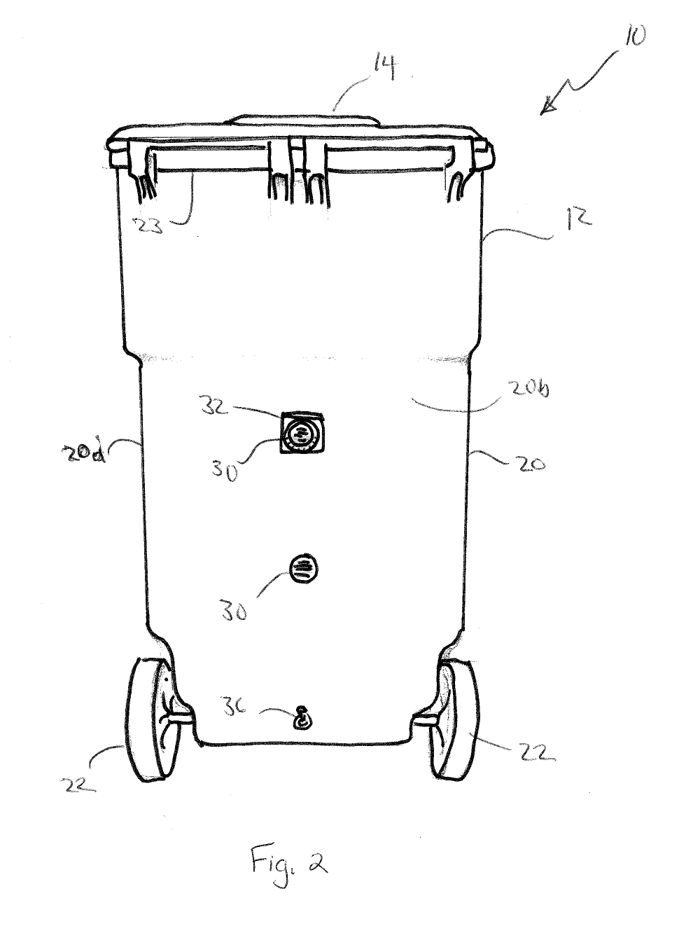

Referring now to FIGS. 1 and 2, a preferred embodiment of the aerated composter and waste collection bin of the present invention is shown generally at 10. The compost bin 10 includes a body portion 12 with a lid 14 and a perforated vent tube 16.

The body portion 12 includes a bottom 18 and four sidewalls 20 (front 20a, rear 20b, right side 20c, and left side) forming a container for material to be composted. The top of the body portion 12 is open to allow material to be loaded into the compost bin 10. The body portion 12 may include wheels 22 (shown in FIG. 2) to permit the compost bin 10 to be easily moved around. Near the top of the body portion 12 are one or more vent structures 21 to allow the exchange of air and vapor form the interior of the body portion 12. The vent structure 21 may be formed integrally with the body portion 12 or be formed as separate structures that are attached to holes bored through the body portion 12. The body portion 12 may also include one or more handles 23 to permit the compost bin 10 to be easily moved.

The lid 14 covers the top of the body portion 12. The lid 14 may include one or more vent structures 24 to allow the exchange of air and vapor from the interior of the body portion 12. The lid 14 prevents rain water from entering the body portion 12. The vent structure 24 may be formed integrally with the lid 14 or be formed as separate structures that are attached to holes bored through the lid 14.

The perforated vent tube 16 extends upwards from the bottom 18 of the body portion 12. The perforated vent tube 16 is anchored to the bottom of the body portion, such as by a flange 26 that is bolted to the bottom 18 of the body portion 12. Liquid from the composting waste material in the body portion 12 is evaporated into the perforated vent tube 16 and vented out of the bin through the vent structures 21, 23 and lateral vent tubes 28, described further below.

The perforated vent tube 16 includes a multitude of perforations formed in it. These perforations may range from 1/16″ to 5/16″. However, 3/16″ perforations have been found to be preferable. The perforations are spaced evenly over the perforated vent tube 16 in intervals of 3/16″. Approximately 33% of the surface area of the perforated vent tube 16 should be perforations to facilitate evaporation of liquids from the composting material. Although round perforations are preferred for ease of manufacture, other shaped perforations may be punched through the perforated vent tube 16, such as oval, square, rectangular, triangular, slits, and other geometric shapes.

One or more lateral vent tubes 28 extend from the perforated vent tube 16 to one or more of the sidewalls 20 of the body portion 12. The lateral vent tubes 28 may include perforations, notches or slits as well. Furthermore, the lateral vent tubes 28 that are located near the bottom 18 of the body portion 12 may include wicking material (not shown) attached thereto, to draw liquid into the lateral vent tubes 28 that pools in the bottom 18 of the body portion 12. The lateral vent tubes 28 are covered by louvered vent openings 30 in the sidewalls 20.

As mentioned above, the surface of the lateral vent tube 28 facing the bottom 18 of the body portion 12 may include additional perforations to facilitate evaporation of liquids from the composting material. These additional perforations may be formed in the same manner as described above for the perforation in the perforated vent tube 16.

Referring back now to FIGS. 1 and 2, to aid in the rapid composting of the waste material in the body portion 12, a fan 32 may be provided at one or more of the lateral vent tubes 28 to circulate air through the lateral vent tube 28, perforated vent tube 16 and body portion 12. The fan 32 is preferably solar powered, but may be battery powered as well or powered through a wired connection. Optionally, the vent fan 32 may be located in the lid 14.

A drain 34 may be included to allow liquid that collects at the bottom 18 of the body portion 12 to be drawn off. The drain 34 may include a stopcock 36 to allow the liquid to be drained at the user's convenience and prevent a mess from forming around the drain 34 of the compost bin 10. Preferably the bottom 18 of the body portion 12 is sloped towards the stopcock 34 to facilitate the removal of the liquid from the compost bin 10.

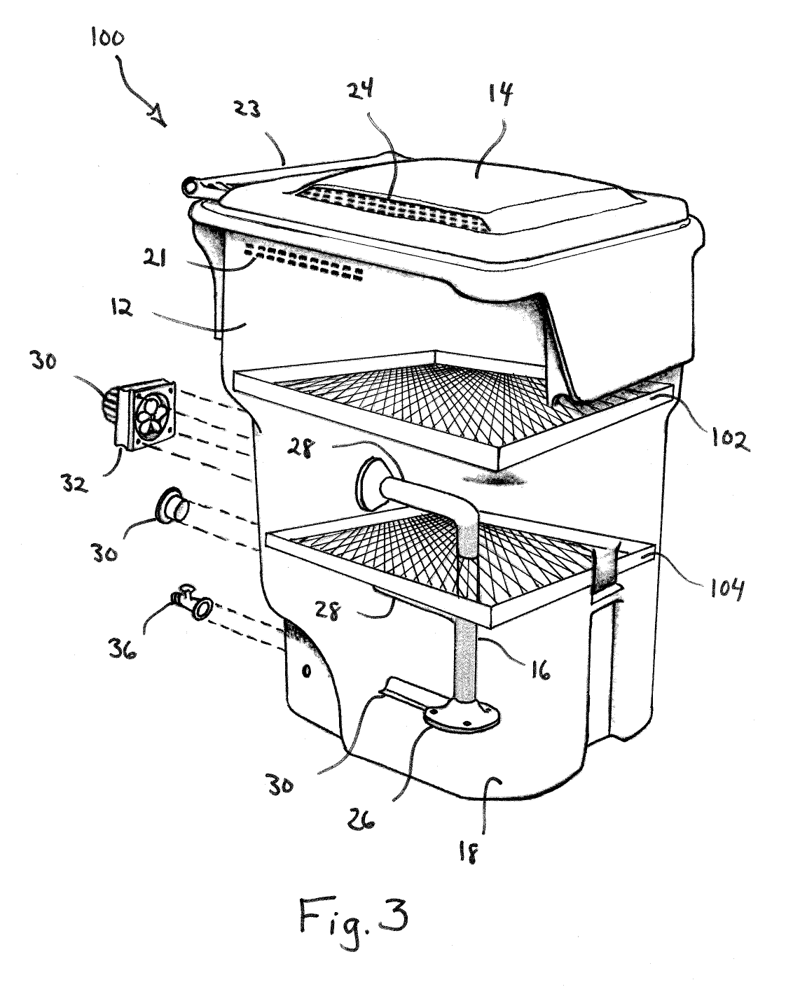

Referring now to FIG. 3, a first alternative embodiment of the compost bin 10 is shown generally at 100. The first alternative embodiment 100 includes an optional first shelf 102 located above the perforated vent tube 16. The first shelf 102 includes a number of openings that function like a grate. That is, the composting material sit atop the first shelf and as the material decays and composts, it falls through the openings to the bottoms 18 below. The large particles of composting material remain above the first shelf 102 until it sufficiently decays enough to fall through the openings of the first shelf.

An optional second shelf 104 may be spaced below the first shelf 102. The second shelf 104 includes a number of openings that are smaller than the openings on the first shelf 102. The second shelf 104 functions in the same manner as the first shelf 102 in that the second shelf 104 functions as a sieve that allows only sufficiently composted materials to reach the bottom 18 of the body portion 12.

Multiple shelves with succeeding smaller openings may be used to sort the composting material. The multiple shelves increase the speed and efficiency of drying the material composting in the body portion by increasing the air flow and evaporative effect of the air circulating through the compost bin 10.

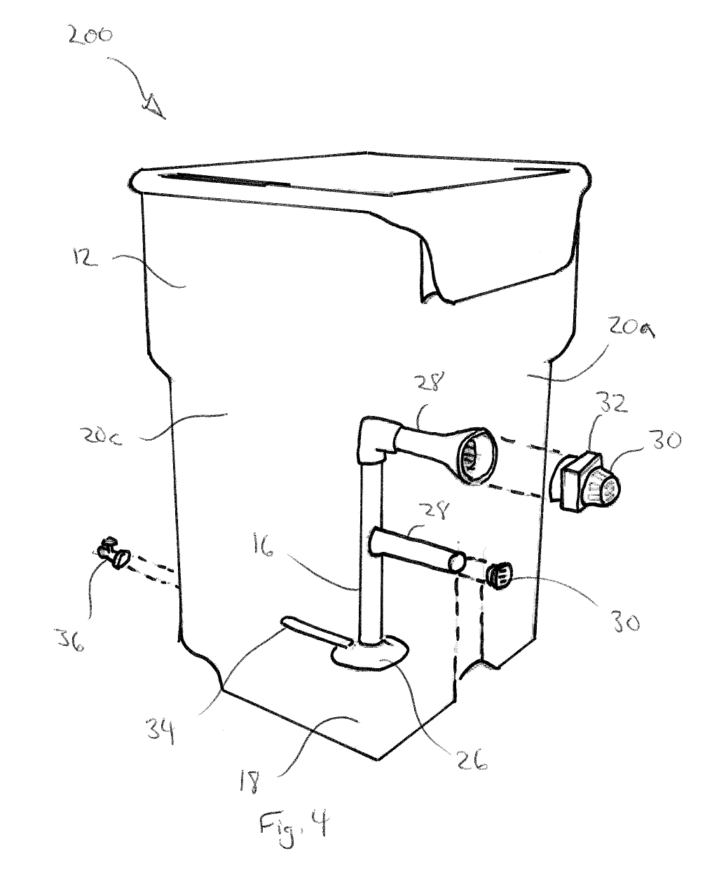

Referring now to FIG. 4, a second alternative embodiment is shown generally at 200. The second alternative embodiment 200 shows that the vent openings 30 may be located on the front 20a of the compost bin 10 in contrast to the preferred embodiment shown in FIGS. 1 and 2, where the vent openings 30 are on the rear 20b. Although not shown specifically, the vent openings 30 may also be located on the sides as well.

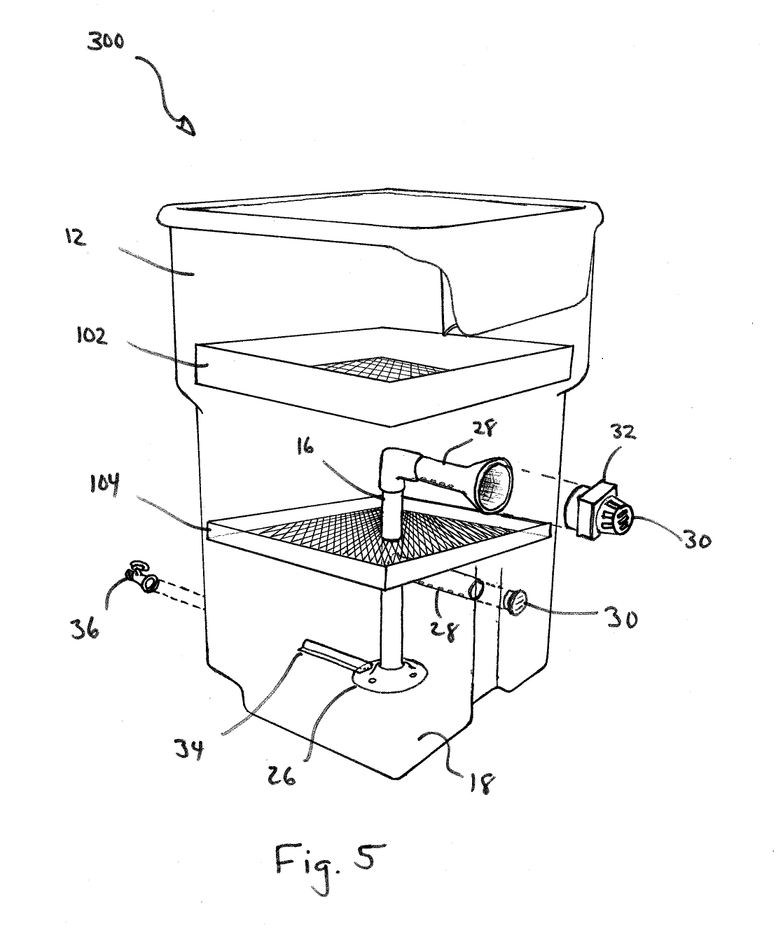

Referring now to FIG. 5, a third alternative embodiment is shown generally at 300. The third alternative embodiment 300 includes features of the first alternative embodiment and second alternative embodiments. Specifically the compost bin includes first and second shelves 102, 104 and forward facing vent openings 30.

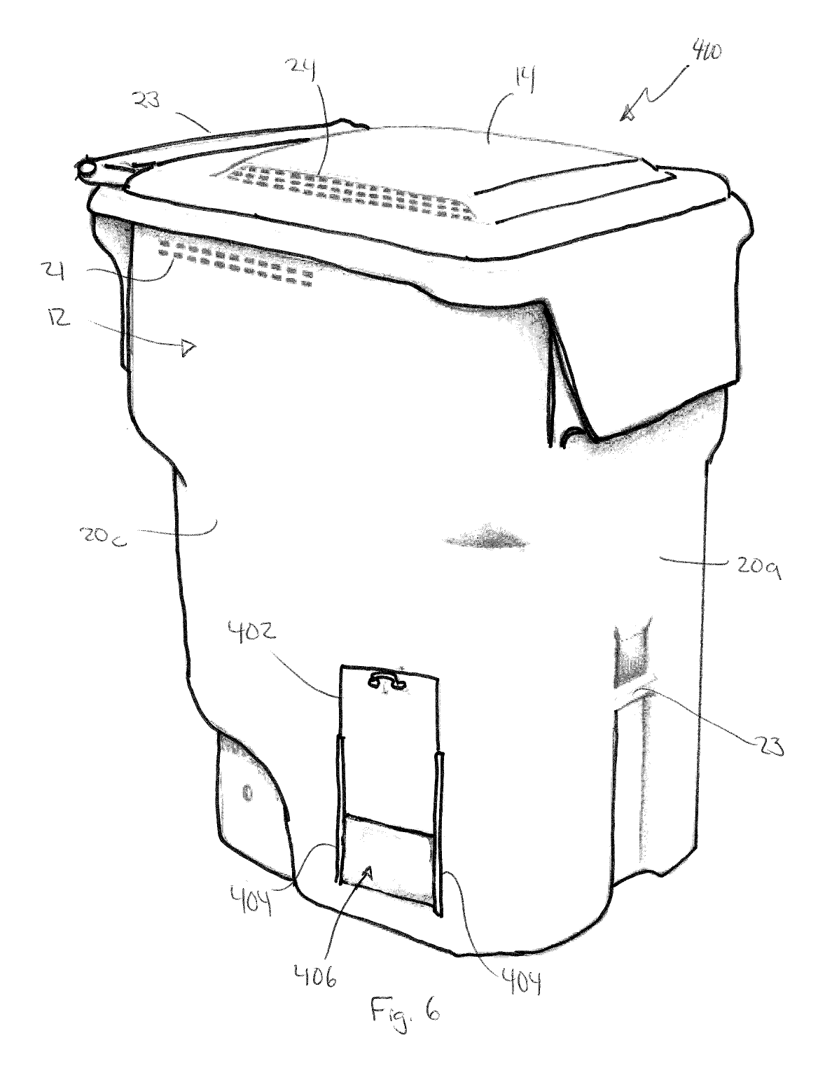

Referring now to FIG. 6, a fourth alternative embodiment is shown generally at 400 that includes an access door 402 on the sidewall 20 of the body portion 12 of the compost bin 10. The access door 402 is fitted within two tracks 404 and slides upwards to reveal an opening 406 into the body portion 12 of the compost bin 10. The opening 406 is preferably located near the bottom 18 of the boy portion 12 to permit the user to easily access fully composted material for use as fertilizer.

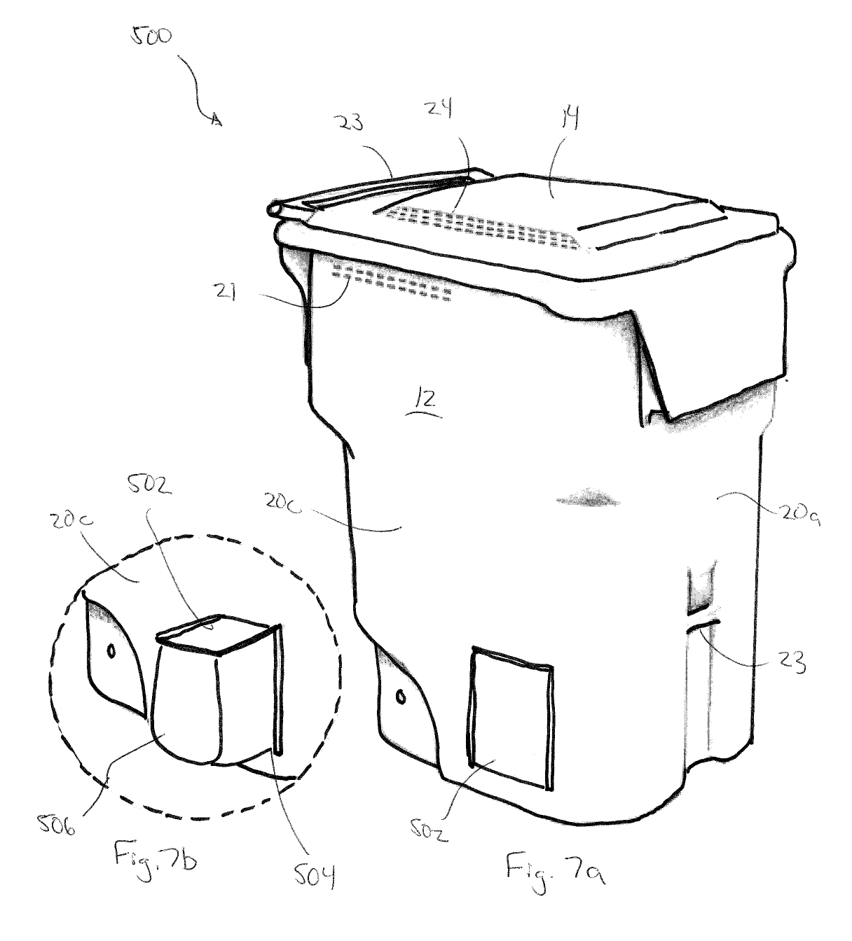

Referring now to FIGS. 7a and 7b, a fifth alternative embodiment of the compost bin is shown generally at 500. In this fifth embodiment 500, the compost bin 10 includes a hinged access hatch 502 that pivots away from the body portion 12 to reveal an opening 504 into the interior of the body portion 12. A basket 506 may be pulled from the opening 504 in the body portion 12. The basket 506 is positioned on the bottom 18 of the body portion 12 and collects fully composted material, while material still composting remains inside the body portion 12 on one of the aforementioned shelves 102,104.

Therefore, it can be seen that the present invention provides a unique solution to the problem of providing a compost bin that minimizes offensive odors and includes the ability to collect compost tea.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A compost bin, comprising:
a body portion having a bottom, at least one sidewall from the bottom, and an open top forming a container;
a perforated tube extending upwards from the bottom of the body portion; and
at least one lateral vent tube connected to the perforated tube and the sidewall of the body portion, said lateral vent tube allowing air to circulate from outside the body portion through to the perforated tube.

2. The compost bin of claim 1, further comprising a drain configured and arranged to allow liquids to drain from the bottom of the body portion.

3. The compost bin of claim 1, further comprising a hinged lid attached to the body portion.

4. The compost bin of claim 3, further comprising at least one vent structure in the lid.

5. The compost bin of claim 1, further comprising a fan configured and arranged to circulate air through the container.

6. The compost bin of claim 5, wherein the fan is connected to the lateral vent tube.

7. The compost bin of claim 1, further comprising at least two wheels attached to the body portion.

8. The compost bin of claim 1, further comprising a louvered vent cover over the lateral vent tube where the lateral vent tube is connected to the sidewall.

9. The compost bin of claim 1, further comprising at least one vent in the sidewall of the body portion.

10. The compost bin of claim 1, further comprising an access door in the sidewall of the body portion.

11. The compost bin of claim 10, further comprising a removable basket configured and arranged to be extracted from the access door.
